(19) United States
(12) Patent Application Publication
(10) Pub. No.: US 2024/0158486 A1
(43) Pub. Date: May 16, 2024

(54) ANTI-N3pGlu AMYLOID BETA ANTIBODIES AND USES THEREOF

(71) Applicant: Eli Lilly and Company, Indianapolis, IN (US)

(72) Inventor: Mark Mintun, Indianapolis, IN (US)

(21) Appl. No.: 18/550,214

(22) PCT Filed: Mar. 11, 2022
(86) PCT No.: PCT/US2022/019903

§ 371 Date: Sep. 12, 2023

Related U.S. Application Data
(60) Provisional application No. 63/192,288, filed on May 24, 2021.
(60) Provisional application No. 63/192,288, filed on May 24, 2021.
(60) Provisional application No. 63/160,490, filed on Mar. 12, 2021.

(51) Int. Cl. C07K 16/18; A61K 39/00

(52) U.S. Cl. CPC C07K 16/18; A61K 2039/505; A61K 2039/545

(57) ABSTRACT

Methods of treating, preventing and/or retarding the progression of cognitive decline, in human subjects having a disease characterized by deposition of Aβ in the brain including Alzheimer's disease, Down's syndrome, and cerebral amyloid angiopathy, using anti-N3pGlu Aβ antibodies.

The present invention relates to the field of medicine. More particularly, the present invention relates to prevention or treatment of a disease characterized by deposition of Amyloid Beta (Aβ) in a human subject, including Alzheimer's disease (AD), Down's syndrome, and cerebral amyloid angiopathy (CAA). Some aspects of the present invention relate to the use of anti-Aβ antibodies, including anti-N3pGlu Aβ antibodies, for the treatment or prevention of a disease characterized by deposition of Aβ. In further aspects, the present invention relates to the treatment or prevention of a disease characterized by deposition of Aβ in human subjects in which the human subjects are selected for treatment or prevention based on their neurological tau level/burden and/or their rate of cognitive decline. In some aspects, the present invention is related to slowing disease progression of AD. In some embodiments, the present invention is related to treatment/prevention/slowing disease progression in patients with evidence of AD neuropathology and either mild cognitive impairment (MCI) or mild dementia stage of AD using the anti-N3pGlu antibodies described herein.

Accumulation of amyloid-β (Aβ) peptide in the form of brain amyloid deposits is an early and essential event in Alzheimer's disease (AD), leading to neurodegeneration and consequently the onset of clinical symptoms: cognitive and functional impairment (Selkoe, “The Origins of Alzheimer Disease: A is for Amyloid,” JAMA 283:1615-7 (2000); Hardy et al., “The Amyloid Hypothesis of Alzheimer's Disease: Progress and Problems on the Road to Therapeutics,” Science 297:353-6 (2002); Masters et al., “Alzheimer's Disease,” Nat. Rev. Dis. Primers 1:15056 (2015); and Selkoe et al., “The Amyloid Hypothesis of Alzheimer's Disease at 25 years,” EMBO Mol. Med. 8:595-608 (2016)). A role for amyloid deposits in driving disease progression is supported by study of uncommon genetic variants that either increase or decrease Aβ deposition (Fleisher et al., “Associations Between Biomarkers and Age in the Presenilin 1 E280A Autosomal Dominant Alzheimer Disease Kindred: A Cross-sectional Study,” JAMA Neurol 72:316-24 (2015); Jonsson et al., “A Mutation in APP Protects Against Alzheimer's Disease and Age-related Cognitive Decline,” Nature 488:96-9 (2012)). In addition, the presence of amyloid deposits early in the disease increases the likelihood of progression of mild cognitive impairment (MCI) to AD dementia (Doraiswamy et al., “Amyloid-β Assessed by Florbetapir F18 PET and 18-month Cognitive Decline: A Multicenter Study,” Neurology 79:1636-44 (2012)). Interventions aiming at removal of Aβ deposits (including amyloid plaques) are hypothesized to slow the clinical progression of AD.

Some known anti-Aβ antibodies include bapineuzumab, gantenerumab, aducanumab, GSK933776, solanezumab, crenezumab, ponezumab, and lecanemab (BAN2401). Antibodies targeting Aβ have shown promise as a therapeutic for Alzheimer's disease in both preclinical and clinical studies. Despite this promise, several antibodies targeting amyloid have failed to meet therapeutic endpoints in multiple clinical trials. The history of anti-amyloid clinical trials spans almost two decades and has, for the most part, cast doubt on the potential of such therapies to effectively treat AD (Aisen et al., “The Future of Anti-amyloid Trials,” The Journal of Prevention of Alzheimer's Disease 7:146-151 (2020)). To date, only a handful of AD treatments have been approved. Utility of such treatment is limited as they only provide partial symptomatic relief and are unable to alter the course of AD progression. See also Budd et al., “Clinical Development of Aducanumab, an Anti-Aβ Human Monoclonal Antibody Being Investigated for the Treatment of Early Alzheimer's Disease,” The Journal of Prevention of Alzheimer's Disease 4(4):255-263 (2017) and Klein et al., “Gantenerumab Reduces Amyloid-β Plaques in Patients with Prodromal to Moderate Alzheimer's Disease: A PET Substudy Interim Analysis,” Alzheimer's Research & Therapy 11.1: 1-12 (2019).

The amyloid deposits found in human patients include a heterogeneous mixture of Aβ peptides. N3pGlu Aβ (also referred to as N3pG Aβ, N3pE Aβ, Aβ pE3-42, or Aβp3-42) is a truncated form of Aβ peptide and is found only in amyloid deposits. N3pGlu Aβ lacks the first two amino acid residues at the N-terminus of human Aβ and has a pyroglutamate which is derived from glutamic acid at the third amino acid position of Aβ. Although N3pGlu Aβ peptide is a minor component of the deposited Aβ in the brain, studies suggest that N3pGlu Aβ peptide has aggressive aggregation properties and accumulates early in the deposition cascade.

Antibodies to Aβ peptide are known in the art, for U.S. Pat. Nos. 7,195,761; 8,591,894; and 8,066,999). Anti-Aβ antibodies to N3pGlu Aβ are known in the art, for example, U.S. Pat. No. 8,679,498 (which is hereby incorporated by reference in its entirety, including the anti-N3pGlu Aβ antibodies disclosed therein) discloses anti-N3pGlu Aβ antibodies and methods of treating diseases, such as, Alzheimer's disease, with the antibodies. Passive immunization by long term chronic administration of antibodies against Aβ, including N3pGlu Aβ, found in deposits has been shown to disrupt the Aβ aggregates and promote the clearance of plaques in the brain in various animal models. Donanemab (disclosed in U.S. Pat. No. 8,679,498) is an antibody directed at the pyroglutamate modification of the third amino acid of amyloid beta (N3pGlu Aβ) epitope that is present only in brain amyloid plaques. The mechanism of action of donanemab is the targeting and removal of existing amyloid plaque, which is a key pathological hallmark of AD.

To date, clinical focus for treatment with donanemab has been specific to early symptomatic AD patients with existing brain amyloid load. However, a second neuropathological hallmark of AD is the presence of intracellular neurofibrillary tangles containing hyperphosphorylated tau protein. Current disease models suggest that Aβ triggers tau pathology, with a more complex and synergistic interaction between Aβ and tau manifesting at later stages and driving disease progression (Busche et al., “Synergy Between Amyloid-β and Tau in Alzheimer's disease,” Nature Neuroscience 23:1183-93 (2020)).

There currently exists no disease-modifying treatment for AD. Thus, a need exists for improved methods of treating diseases, including AD, characterized by deposition of Aβ in a human subject. Such methods should aid in identifying patients based on whether such patient is likely to have a therapeutic benefit from such treatment. Such treatments and methods should further not be attendant upon increased cytotoxicity or other known adverse events. The present invention meets one of more of these needs.

Doody et al., “Phase 3 Trials of Solanezumab for Mild-to-Moderate Alzheimer's Disease,” NEJM, 370; 4, 311-321 (2014) indicate that “[n]o clear differential treatment effects on efficacy measures were observed between APOE ε4 carriers and noncarriers.” It has now been found that administering an anti-N3pGlu Aβ antibody to a human subject that has one or two alleles of APOE e4 (e.g., a carrier of APOE e4) provides unexpected and surprising efficacy when compared to non-carriers of one or more of those alleles. Thus, the present embodiments involve administering doses of anti-N3pGlu Aβ antibodies to patients who have that allele as a means of slowing the cognitive decline of those patients. Specifically, it has been found that there is a greater effect in carriers of APOE e4 than in non-carriers, when the patients are administered anti-N3pGlu Aβ antibodies. This means that the patients that have APOE e4 have less cognitive decline than non-carriers, when measured using various clinical measurements and at various endpoints.

According to embodiments, the present invention provides methods of treating or preventing a disease characterized by amyloid beta (Aβ) deposits in the brain of a human subject who has been determined to have a high neurological tau burden, comprising administering a therapeutically effective amount of an anti-Aβ antibody. Additionally, according to particular embodiments, the present invention provides methods of treating or preventing a disease characterized by Aβ deposits in the brain of a human subject who has been determined to have a posterior-lateral temporal lobe tau burden, comprising administering a therapeutically effective amount of an anti-Aβ antibody.

According to particular embodiments, the present invention provides methods of treating or preventing a disease characterized by amyloid beta (Aβ) deposits in the brain of a human subject who has been determined to have a high neurological tau burden and having one or two alleles of epsilon-4 allele of apolipoprotein E (referred to herein as APOE e4 or APOE4), comprising administering a therapeutically effective amount of an anti-Aβ antibody. Additionally, according to particular embodiments, the present invention provides methods of treating or preventing a disease characterized by Aβ deposits in the brain of a human subject who has been determined to have a posterior-lateral temporal lobe tau burden, comprising administering a therapeutically effective amount of an anti-Aβ antibody.

According to some embodiments, the present invention provides an anti-Aβ antibody for use in the treatment or prevention of a disease characterized by Aβ deposits in the brain of a human subject who has been determined to have a high neurological tau burden, comprising administering a therapeutically effective amount of an anti-Aβ antibody. In some embodiments, the human subject has been determined to have a high neurological tau burden as well as having one or two alleles of APOE e4.

In some embodiments, the present invention provides an anti-Aβ antibody for use in the treatment or prevention of a disease characterized by Aβ deposits in the brain of a human subject who has been determined to have a posterior-lateral temporal lobe tau burden. In some embodiments, the human subject has been determined to have a posterior-lateral temporal lobe tau burden as well as having one or two alleles of APOE e4.

Additionally, in some embodiments, the present invention provides an anti-Aβ antibody for use in treating, preventing, or retarding the progression of Alzheimer's Disease (AD) in a human subject who has been determined to have slow progressing AD cognitive decline. Some embodiments of the present invention provide an anti-Aβ antibody for use in treating, preventing, or retarding the progression of Alzheimer's Disease (AD) in a human subject who has been determined to have slow progressing AD cognitive decline and one or two alleles of APOE e4.

Further, according to some embodiments, the present invention provides the use of an anti-Aβ antibody in the manufacture of a medicament for treatment or prevention of a disease characterized by Aβ deposits in the brain of a human subject who has been determined to have a i) high neurological tau burden or ii) high neurological tau burden and one or two alleles of APOE e4. In some embodiments, the present invention provides for the use of an anti-Aβ antibody in the manufacture of a medicament for treatment or prevention of a disease characterized by Aβ deposits in the brain of a human subject who has been determined to have i) a posterior-lateral temporal lobe tau burden or ii) a posterior-lateral temporal lobe tau burden and one or two alleles of APOE e4. And in further embodiments, the present invention provides for the use of an anti-Aβ antibody in the manufacture of a medicament for treating, preventing, or retarding the progression of Alzheimer's Disease (AD) in a human subject who has been determined to have i) slow progressing AD cognitive decline or ii) one or two alleles of APOE e4 and slow progressing AD cognitive decline.

According to some of the embodiments provided herein, the human subject has been determined to have posterior-lateral temporal lobe and occipital lobe tau burden. In some embodiments, the human subject has been determined to have posterior-lateral temporal lobe, occipital lobe, and parietal lobe tau burden. In some embodiments, the human subject has been determined to have posterior-lateral temporal lobe, occipital lobe, parietal lobe, and frontal lobe tau burden. In some embodiments, the human subject has been determined to have one or more of posterior-lateral temporal lobe, occipital lobe, parietal lobe and/or frontal lobe tau burden by neurological PET imaging. In some embodiments, the one or more of posterior-lateral temporal lobe, occipital lobe, parietal lobe and/or frontal lobe tau burden corresponds a neurological tau burden of greater than 1.46 SUVr.

According to some of the embodiments provided herein, the human subject has been determined to have one or two alleles of APOE e4 and posterior-lateral temporal lobe and occipital lobe tau burden. In some embodiments, the human subject has been determined to have one or two alleles of APOE e4 and posterior-lateral temporal lobe, occipital lobe, and parietal lobe tau burden. In some embodiments, the human subject has been determined to have one or two alleles of APOE e4 and posterior-lateral temporal lobe, occipital lobe, parietal lobe, and frontal lobe tau burden. In some embodiments, the human subject has been determined to have one or more of posterior-lateral temporal lobe, occipital lobe, parietal lobe and/or frontal lobe tau burden by neurological PET imaging and one or two alleles of APOE e4. In some embodiments, the one or more of posterior-lateral temporal lobe, occipital lobe, parietal lobe and/or frontal lobe tau burden corresponds a neurological tau burden of greater than 1.46 SUVr.

According to additional embodiments, the present invention provides methods of treating, preventing, or retarding the progression of Alzheimer's Disease (AD) in a human subject who has been determined to have slow progressing AD cognitive decline, comprising administering a therapeutically effective amount of an anti-Aβ antibody. According to some embodiments, the human subject has been determined to have a high neurological tau burden. According to some embodiments, the human subject has been determined to have one or two alleles of APOE e4. In some embodiments, the human subject has been determined to have posterior-lateral temporal lobe tau burden. In some embodiments, the human subject has been determined to have posterior-lateral temporal lobe and occipital lobe tau burden. In some embodiments, the human subject has been determined to have posterior-lateral temporal lobe, occipital lobe, and parietal lobe tau burden. In some embodiments, the human subject has been determined to have posterior-lateral temporal lobe, occipital lobe, parietal lobe, and frontal lobe tau burden. In some embodiments, the human subject has been determined to have posterior-lateral temporal lobe tau burden and one or two alleles of APOE e4. In some embodiments, the human subject has been determined to have one or two alleles of APOE e4 and posterior-lateral temporal lobe and occipital lobe tau burden. In some embodiments, the human subject has been determined to have one or two alleles of APOE e4 and posterior-lateral temporal lobe, occipital lobe, and parietal lobe tau burden. In some embodiments, the human subject has been determined to have one or two alleles of APOE e4 and posterior-lateral temporal lobe, occipital lobe, parietal lobe, and frontal lobe tau burden.

According to embodiments of the present invention provided herein, the human subject has been determined to have slow progressing AD cognitive decline by one of more of ADAS-Cog, iADL, CDR-SB, MMSE, APOE-4 genotyping, tau levels, P-tau levels. and/or iADRS. In some embodiments, the human subject has been determined to have slow progressing AD cognitive decline by iADRS. In some embodiments, iADRS has declined by less than 20. In some embodiments, iADRS has declined by less than 20 over a 6-month period. In some embodiments, iADRS has declined by less than 20 over a 12-month period. In some embodiments, iADRS has declined by less than 20 over an 18-month period. In some embodiments, iADRS has declined by less than 20 over a 24-month period. In some embodiments, the human subject has been determined to have slow progressing AD cognitive decline by APOE-4 genotyping. In some embodiments, the human subject has been determined to be APOE-4 heterozygous. In some embodiments, the human subject has been determined to be APOE-4 homozygous negative. In some embodiments, the human subject has been determined to have slow progressing AD cognitive decline by MMSE. In some embodiments, the human subject has been determined to have MMSE of above 27. In some embodiments, MMSE has declined by less than 3. In some embodiments, MMSE has declined by less than 3 over a 6-month period. In some embodiments, MMSE has declined by less than 3 over a 12-month period. In some embodiments, MMSE has declined by less than 3 over an 18-month period. In some embodiments, MMSE has declined by less than 3 over a 24-month period.

According to embodiments of the present invention provided herein, the human subject has been determined to have a high neurological tau burden by neurological PET imaging. In some embodiments, the human subject has been determined to have high neurological tau burden by neurological PET imaging above 1.46 SUVr. In some embodiments, the human subject has been determined to have high neurological tau burden by quantification of human tau phosphorylated at threonine at residue 217 (“hTau-pT217”). In some embodiments, hTau-pT217 is quantified in a biological sample of the human subject. In some embodiments, the biological sample is cerebral spinal fluid. In some embodiments, the biological sample is one of blood, plasma, or serum.

According to embodiments of the present invention provided herein, the anti-Aβ antibody comprises an anti-N3pG Aβ antibody. In some embodiments, the anti-N3pG Aβ antibody comprises a light chain variable region (LCVR) and a heavy chain variable region (HCVR), wherein the LCVR and HCVR are selected from: (a) the LCVR comprising the amino acid sequence of SEQ ID NO: 1 and the HCVR comprising the amino acid sequence of SEQ ID NO: 2; or (b) the LCVR comprising the amino acid sequence having at least 95% homology to the amino acid sequence of SEQ ID NO: 1 and the HCVR comprising the amino acid sequence having at least 95% homology to the amino acid sequence of SEQ ID NO: 2.

According to some embodiments of the invention provided herein, administering the anti-N3pG Aβ antibody comprises: i) administering to the human subject one or more first doses of about 100 mg to about 700 mg of an anti-N3pG Aβ antibody, wherein each first dose is administered once about every four weeks; and ii) about four weeks after administering the one or more first doses, administering to the human subject one or more second doses of greater than 700 mg to about 1400 mg of the anti-N3pG Aβ antibody, wherein each second dose is administered once about every 4 weeks. In some embodiments, the human subject is administered the first dose once, two times, or three times before administering the second dose. In some embodiments, the human subject is administered first doses of about 700 mg. In some embodiments, the human subject is administered one or more second doses of about 800 mg, about 900 mg, about 1000 mg, about 1100 mg, about 1200 mg, about 1300 mg, or about 1400 mg. In some embodiments, the human subject is administered one or more second doses of about 1400 mg. In some embodiments, the anti-N3pGlu Aβ antibody is administered to the human subject for a duration of up to 72 weeks.

According to embodiments of the present invention provided herein, the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy. In some embodiments, the human subject is an early symptomatic AD patient. In some embodiments, the human subject has prodromal AD and/or mild dementia due to AD.

For the purposes of the present invention, the tau level or burden (as used interchangeably herein) of a human subject can be determined using techniques or methods that, e.g., detect or quantitate i) neurological or brain tau deposition, ii) tau in blood, serum and/or plasma, or iii) tau in cerebrospinal fluid. In some embodiments, neurological tau burden (whether determined via PET or via a blood, serum, plasma, or cerebrospinal fluid assay) can be used to stratify subjects based on neurological tau burden (e.g., low, moderate, or high neurological tau burden).

Neurological tau burden can be determined using methods, such as, tau imaging with radiolabeled PET compounds (Leuzy et al., “Diagnostic Performance of RO948 F18 Tau Positron Emission Tomography in the Differentiation of Alzheimer Disease from Other Neurodegenerative Disorders,” JAMA Neurology 77.8:955-965 (2020); Ossenkoppele et al., “Discriminative Accuracy of [¹⁸F]-flortaucipir Positron Emission Tomography for Alzheimer Disease vs Other Neurodegenerative Disorders,” JAMA 320, 1151-1162, doi:10.1001/jama.2018.12917 (2018), which are hereby incorporated by reference in their entireties) including [¹⁸F]-florbtaucipir, which is a PET ligand. PET tau images can be, for example, quantitatively evaluated to estimate an SUVr (standardized uptake value ratio) by published methods (Pontecorvo et al., “A Multicentre Longitudinal Study of Flortaucipir (18F) in Normal Ageing, Mild Cognitive Impairment and Alzheimer's Disease Dementia,” Brain 142:1723-35 (2019); Devous et al., “Test-Retest Reproducibility for the Tau PET Imaging Agent Flortaucipir F18,” Journal of Nuclear Medicine 59:937-43 (2018); Southekal et al., “Flortaucipir F18 Quantitation Using Parametric Estimation of Reference Signal Intensity,” J. Nucl. Med. 59:944-51 (2018), which are hereby incorporated by reference in their entireties) and/or to visually evaluate patients, e.g., to determine whether the patient has an AD pattern (Fleisher et al., “Positron Emission Tomography Imaging With [¹⁸F]-flortaucipir and Postmortem Assessment of Alzheimer Disease Neuropathologic Changes,” JAMA Neurology 77:829-39 (2020), which is hereby incorporated by reference in its entirety). Lower SUVr values indicate less tau burden while higher SUVr values indicate a higher tau burden. In an embodiment, quantitative assessment by a flortaucipir scan is accomplished through an automated image processing pipeline as described in Southekal et al., “Flortaucipir F18 Quantitation Using Parametric Estimation of Reference Signal Intensity,” J. Nucl. Med. 59:944-951 (2018), which is hereby incorporated by reference in its entirety. In some embodiments, counts within a specific target region of interest in the brain (e.g., multiblock barycentric discriminant analysis or MUBADA, see Devous et al, “Test-Retest Reproducibility for the Tau PET Imaging Agent Flortaucipir F18,” J. Nucl. Med. 59:937-943 (2018), which is hereby incorporated by reference in its entirety) are compared with a reference region wherein the reference region is, e.g., whole cerebellum, (wholeCere), cerebellar GM (cereCrus), atlas-based white matter (atlasWM), subject-specific WM (ssWM, e.g., using parametric estimate of reference signal intensity (PERSI), see Southekal et al., “Flortaucipir F18 Quantitation Using Parametric Estimation of Reference Signal Intensity,” J. Nucl. Med 59:944-951 (2018), which is hereby incorporated by reference in its entirety). An exemplary method of determining tau burden is a quantitative analysis reported as a standardized uptake value ratio (SUVr), which represents counts within a specific target region of interest in the brain (e.g., MUBADA,) when compared with a reference region (e.g., using PERSI).

In some embodiments, phosphorylated tau (P-tau; either phosphorylated at threonine 181 or 217, or a combination thereof) can be used to measure the tau load/burden for the purposes of the present invention (Barthelemy et al., “Cerebrospinal Fluid Phospho-tau T217 Outperforms T181 as a Biomarker for the Differential Diagnosis of Alzheimer's Disease and PET Amyloid-positive Patient Identification,” Alzheimer's Res. Ther. 12, 26, doi:10.1186/s13195-020-00596-4 (2020); Mattsson et al., “Aβ Deposition is Associated with Increases in Soluble and Phosphorylated Tau that Precede a Positive Tau PET in Alzheimer's Disease,” Science Advances 6, eaaz2387 (2020), which are hereby incorporated by reference their entireties). In a particular embodiment, antibodies directed against human tau phosphorylated at threonine at residue 217 can be used to measure the tau load/burden in a subject (see International Patent Application Publication No. WO 2020/242963, which is incorporated by reference in its entirety). The present invention includes, in some embodiments, the use of anti-tau antibodies disclosed in WO 2020/242963 to measure the tau load/burden in a subject. Anti-tau antibodies disclosed in WO 2020/242963 are directed against isoforms of human tau expressed in the CNS (e.g., recognizing the isoforms expressed in the CNS and not recognizing isoforms of human tau expressed exclusively outside the CNS).

A subject is positive for amyloid deposits when amyloid is detected in the brain by methods such as, amyloid imaging with radiolabeled PET compounds or using a diagnostic that detects Aβ or a biomarker for Aβ. Exemplary methods that can be used to measure the brain amyloid load/burden include, e.g., Florbetapir (Carpenter, et al., “The Use of the Exploratory IND in the Evaluation and Development of ¹⁸F-PET Radiopharmaceuticals for Amyloid Imaging in the Brain: A Review of One Company's Experience,” The Quarterly Journal of Nuclear Medicine and Molecular Imaging 53.4:387 (2009), which is hereby incorporated by reference in its entirety); Florbetaben (Syed et al., “[¹⁸F]Florbetaben: A Review in β-Amyloid PET Imaging in Cognitive Impairment,” CNS Drugs 29, 605-613 (2015), which is hereby incorporated by reference in its entirety); and Flutemetamol (Heurling et al., “Imaging β-amyloid Using [¹⁸F] Flutemetamol Positron Emission Tomography: From Dosimetry to Clinical Diagnosis,” European Journal of Nuclear Medicine and Molecular Imaging 43.2: 362-373 (2016), which is hereby incorporated by reference in its entirety). [¹⁸F]-florbetapir can provide a qualitative and quantitative measurement of brain plaque load in patients, including patients with prodromal AD or mild AD dementia and can be used to assess amyloid plaque reductions from the brain as well.

Additionally, cerebrospinal fluid or plasma-based analysis of β-amyloid can also be used to measure the amyloid load/burden. For example, Aβ42 can be used to measure brain amyloid (Palmqvist, S. et al., “Accuracy of Brain Amyloid Detection in Clinical Practice Using Cerebrospinal Fluid Beta-amyloid 42: a Cross-validation Study Against Amyloid Positron Emission Tomography. JAMA Neurol 71, 1282-1289 (2014), which is hereby incorporated by reference in its entirety). In some embodiments, the ratio of Aβ42/Aβ40 or Aβ42/Aβ38 can be used as a biomarker for amyloid beta (Janelidze et al., “CSF Abeta42/Abeta40 and Abeta42/Abeta38 Ratios: Better Diagnostic Markers of Alzheimer Disease,” Ann Clin Transl Neurol 3, 154-165 (2016), which is hereby incorporated by reference in its entirety). In some embodiments, deposited brain amyloid plaque or Aβ in CSF or plasma can be used to stratify subjects into groups based on amyloid load/burden.

As used herein, “anti-N3pGlu Aβ antibody,” “anti-N3pG antibody,” or “anti-N3pE antibody,” used interchangeably, refer to an antibody that binds preferentially to N3pGlu Aβ over Aβ1-40 or Aβ1-42. One of ordinary skill in the art will appreciate and recognize that “anti-N3pGlu Aβ antibody”, and several specific antibodies, including, “hE8L”, “B 12L” and “R17L” are identified and disclosed (along with methods for making and using such antibodies) in U.S. Pat. No. 8,679,498 B2 (which is hereby incorporated by reference in its entirety). See, for example, Table 1 of U.S. Pat. No. 8,679,498 B2. Each of the antibodies disclosed in U.S. Pat. No. 8,679,498 B2, including “hE8L”, “B 12L” and “R17L” antibodies, may be used as the anti-N3pGlu Aβ antibody of the present invention or in place of the anti-N3pGlu Aβ antibodies described in various aspects of the present invention. Other representative species of an anti-N3pGlu Aβ antibody include, but are not limited to, antibodies disclosed U.S. Pat. Nos. 8,961,972; 10,647,759; 9,944,696; WO 2010/009987A2; WO 2011/151076A2; WO 2012/136552A1 and equivalents thereto, e.g., under 35 U.S.C 112(f).

One of ordinary skill in the art will appreciate and recognize that “anti-N3pGlu Aβ antibody”, and several specific antibodies are identified and disclosed (along with methods for making and using such antibodies) in U.S. Pat. No. 8,961,972 (which is hereby incorporated by reference in its entirety); U.S. Pat. No. 10,647,759 (which is hereby incorporated by reference in its entirety); and U.S. Pat. No. 9,944,696 (which is hereby incorporated by reference in its entirety). Any of the anti-N3pGlu Aβ antibodies disclosed in the U.S. Pat. Nos. 8,961,972; 9,944,696; and 10,647,759 may be used as the anti-N3pGlu Aβ antibody of the present invention or in place of the anti-N3pGlu Aβ antibodies described in various aspects of the present invention.

One of ordinary skill in the art will appreciate and recognize that “anti-N3pGlu Aβ antibody”, and several specific antibodies, including, “Antibody VI”, “Antibody VII”, “Antibody VIII”, and “Antibody IX” are identified and disclosed (along with methods for making and using such antibodies) in WO2010/009987A2 (which is hereby incorporated by reference in its entirety). Each of these four antibodies (e.g., “Antibody VI”, “Antibody VII”, “Antibody VIII”, and “Antibody IX”) may be used as the anti-N3pGlu Aβ antibody of the present invention or in place of the anti-N3pGlu Aβ antibodies described in various aspects of the present invention.

One of ordinary skill in the art will appreciate and recognize that “anti-N3pGlu Aβ antibody”, and several specific antibodies, including, “Antibody X” and “Antibody XI” are identified and disclosed (along with methods for making and using such antibodies) in WO 2011/151076A2 (which is hereby incorporated by reference in its entirety). Each of these two antibodies (e.g., “Antibody X” and “Antibody XI”) may be used as the anti-N3pGlu Aβ antibody of the present invention or in place of the anti-N3pGlu Aβ antibodies described in various aspects of the present invention.

One of ordinary skill in the art will appreciate and recognize that “anti-N3pGlu Aβ antibody”, and several specific antibodies, including, “Antibody XII” and “Antibody XIII” are identified and disclosed (along with methods for making and using said antibodies) in WO 2012/136552A1 (which is hereby incorporated by reference in its entirety). Each of these two antibodies (e.g., “Antibody XII” and “Antibody XIII”) may be used as the anti-N3pGlu Aβ antibody of the present invention or in place of the anti-N3pGlu Aβ antibodies described in various aspects of the present invention.

As used herein, an “antibody” is an immunoglobulin molecule comprising two HC and two LC interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions. Assignment of amino acids to CDR domains within the LCVR and HCVR regions of the antibodies of the present invention is based on the following: Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)). Following the above method, the CDRs of the antibodies of the present invention were determined.

The antibodies of the present invention are monoclonal antibodies (“mAbs”). Monoclonal antibodies can be produced, for example, by hybridoma technologies, recombinant technologies, phage display technologies, synthetic technologies, e.g., CDR-grafting, or combinations of such or other technologies known in the art. The monoclonal antibodies of the present invention are human or humanized. Humanized antibodies can be engineered to contain one or more human framework regions (or substantially human framework regions) surrounding CDRs derived from a non-human antibody. Human framework germline sequences can be obtained from ImunoGeneTics (INGT) via their website, http://imgt.cines.fr, or from The Immunoglobulin FactsBook by Marie-Paule Lefranc and Gerard Lefranc, Academic 25 Press, 2001, ISBN 012441351. Techniques for generating human or humanized antibodies are well known in the art. In another embodiment of the present invention, the antibody, or the nucleic acid encoding the same, is provided in isolated form. As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

Antibodies of the present invention are administered as a pharmaceutical composition. The pharmaceutical composition comprising an antibody of the present invention can be administered to a subject at risk for, or exhibiting, diseases or disorders as described herein by parental routes (e.g., subcutaneous, intravenous, intraperitoneal, intramuscular). Subcutaneous and intravenous routes are preferred.

The terms “treatment,” “treating” or “to treat” and the like include restraining, slowing, or stopping the progression or severity of an existing symptom, condition, disease, or disorder in a subject. The term “subject” refers to a human.

The term “prevention” means prophylactic administration of the antibody of the present invention to an asymptomatic subject or a subject with pre-clinical Alzheimer's disease to prevent onset or progression of the disease.

The term “retarding the progression of” as used herein means delaying or holding back the progression of a disease or symptom thereof in a subject.

The terms “disease characterized by deposition of Aβ” or a “disease characterized by Aβ deposits” are used interchangeably and refer to a disease that is pathologically characterized by Aβ deposits in the brain or in brain vasculature. This includes diseases such as Alzheimer's disease, Down's syndrome, and cerebral amyloid angiopathy. A clinical diagnosis, staging or progression of Alzheimer's disease can be readily determined by the attending diagnostician or health care professional, as one skilled in the art, by using known techniques and by observing results. This generally includes brain plaque imaging, mental or cognitive assessment (e.g., Clinical Dementia Rating—summary of boxes (CDR-SB), Mini-Mental State Exam (MMSE) or Alzheimer's Disease Assessment Scale-Cognitive (ADAS-Cog)) or functional assessment (e.g., Alzheimer's Disease Cooperative Study-Activities of Daily Living (ADCS-ADL). The cognitive and functional assessment can be used to determine changes in a patient's cognition (e.g., cognitive decline) and function (e.g., functional decline). Accordingly, a subject may be determined to have a “slow progressing” cognitive decline according to a technique as described herein. In an exemplary embodiment, a “slow progressing” cognitive decline may be identified by iADRS wherein a subject's iADRs has declined by less than about 20, for example over a given period of time (e.g., 6, 12, 18 or 24 months). In another exemplary embodiment, a “slow progressing” cognitive decline may be identified by APOE-4 genotyping wherein a subject is APOE-4 homozygous negative or APOE-4 heterozygous. In another exemplary embodiment, a “slow progressing” cognitive decline may be identified by MMSE, wherein the subject has been determined to have a MMSE of about 27 or a MMSE decline of less than about 3 over a given period of time (e.g., 6, 12, 18 or 24 months). “Clinical Alzheimer's disease” as used herein is a diagnosed stage of Alzheimer's disease. It includes conditions diagnosed as prodromal Alzheimer's disease, mild Alzheimer's disease, moderate Alzheimer's disease, and severe Alzheimer's disease. The term “pre-clinical Alzheimer's disease” is a stage that precedes clinical Alzheimer's disease, where measurable changes in biomarkers (such as CSF Aβ42 levels or deposited brain plaque by amyloid PET) indicate the earliest signs of a patient with Alzheimer's pathology, progressing to clinical Alzheimer's disease. This is usually before symptoms such as memory loss and confusion are noticeable. Pre-clinical Alzheimer's disease also includes pre-symptomatic autosomal dominant carriers, as well as patients with higher risk for developing AD by virtue of carrying one or two APOE e4 alleles.

A reduction or slowing of cognitive decline can be measured by cognitive assessments such as Clinical Dementia Rating—summary of boxes, Mini-Mental State Exam or Alzheimer's Disease Assessment Scale-Cognitive. A reduction or slowing of functional decline can be measured by functional assessments such as ADCS-ADL.

As used herein, “mg/kg” means an amount, in milligrams, of antibody or drug administered to a subject based on his or her bodyweight in kilograms. A dose is given at one time. For example, a 10 mg/kg dose of antibody for a subject weighing 70 kg would be a single 700 mg dose of antibody given in a single administration. Similarly, a 20 mg/kg dose of antibody for a subject weighing 70 kg would be a 1400 mg dose of antibody given at a single administration.

As used herein, a human subject has “very low tau” burden if the tau burden is less than 1.10 SUVr (<1.10 SUVr) using ¹⁸F-flortaucipir based quantitative analysis where quantitative analysis refers to calculation of SUVr and SUVr represents counts within a specific target region of interest in the brain (multiblock barycentric discriminant analysis or MUBADA, see Devous et al, “Test-Retest Reproducibility for the Tau PET Imaging Agent Flortaucipir F18,” J. Nucl. Med. 59:937-943 (2018)) when compared with a reference region (parametric estimate of reference signal intensity or PERSI, see, Southekal et al., “Flortaucipir F 18 Quantitation Using Parametric Estimation of Reference Signal Intensity,” J. Nucl. Med 59:944-951 (2018)). As used herein, a human subject has “very low tau to moderate tau” burden if the tau burden is less than or equal to 1.46 SUVr (i.e., ≤1.46 SUVr) using 18F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med 59:944-951 (2018)).

As used herein, a human subject has “low tau to moderate tau” burden if the tau burden is from greater than or equal to 1.10 to less than or equal to 1.46 (i.e., ≤1.10 SUVr to ≤1.46 SUVr) using ¹⁸F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med. 59:944-951 (2018)). A human subject having “low tau to moderate tau” burden can also be referred to as having “intermediate” tau burden.

As used herein, a human subject has “high tau” burden if the tau burden is greater than 1.46 SUVr (i.e., >1.46 SUVr) using ¹⁸F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med 59:944-951 (2018)).

As used herein, the term “about” means up to ±10%.

The terms “human subject” and “patient” are used interchangeably in the present disclosure.

As used herein, “methods of treatment” are equally applicable to use of a composition for treating the diseases or disorders described herein and/or compositions for use and/or uses in the manufacture of a medicaments for treating the diseases or disorders described herein.

The following Examples further illustrate the present invention. It should be understood however, that the Examples are set forth by way of illustration and not limitation, and that various modifications may be made by one of ordinary skill in the art.

### EXAMPLES

### Example 1: Expression and Purification of Engineered N3pGlu Aβ Antibodies

Antibodies to N3pGlu Aβ are known in the art. For example, U.S. Pat. Nos. 8,679,498 and 8,961,972 (which are hereby incorporated by reference in their entireties) disclose anti-N3pGlu Aβ antibodies, method of making antibodies, antibody formulations, and methods of treating diseases, such as, Alzheimer's disease with such antibodies.

An exemplary method of expressing and purifying anti-N3pGlu Aβ antibodies of the present invention is as follows. An appropriate host cell, such as HEK 293 EBNA or CHO, is either transiently or stably transfected with an expression system for secreting antibodies using an optimal predetermined HC:LC vector ratio or a single vector system encoding both HC and LC. Clarified media, into which the antibody has been secreted, is purified using any of many commonly used techniques. For example, the medium may be conveniently applied to a Protein A or G Sepharose FF column that has been equilibrated with a compatible buffer, such as phosphate buffered saline (pH 7.4). The column is washed to remove nonspecific binding components. The bound antibody is eluted, for example, by pH gradient (such as 0.1 M sodium phosphate buffer pH 6.8 to 0.1 M sodium citrate buffer (pH 2.5). Antibody fractions are detected, such as by SDS-PAGE, and are pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, or hydroxyapatite chromatography. The purity of the antibody after these chromatography steps is greater than 99%. The product may be immediately frozen at −70° C. or may be lyophilized. The amino acid sequences for the anti-N3pGlu Aβ antibodies are provided in the sequence listings.

### Example 2: Comparison of Neurological Tau Burden with Cognitive Change

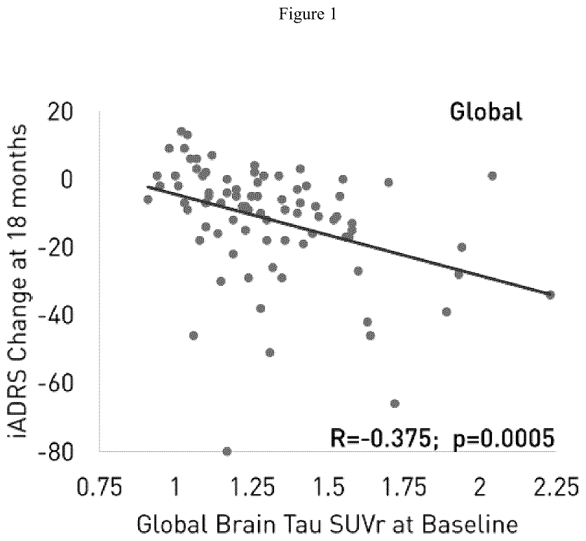

Assessment of neurological tau burden, both global and frontal lobe, in comparison to cognitive change is measured substantially as described below. Subjects are assessed for neurological tau burden, both global and frontal lobe, by flortaucipir as described herein at baseline. Additionally, at baseline, subjects are cognitively assessed under one of iADRS or CDR-SB, as known in the art. Subjects may be cognitively reassessed, for example, under one of iADRS or CDR-SB, at a given point in time thereafter, for example, at 26 weeks, 52 weeks, 78 weeks, or 104 weeks. Change in cognitive assessment versus neurological tau burden may be plotted as set forth in FIGS. 1, 2 and 3.

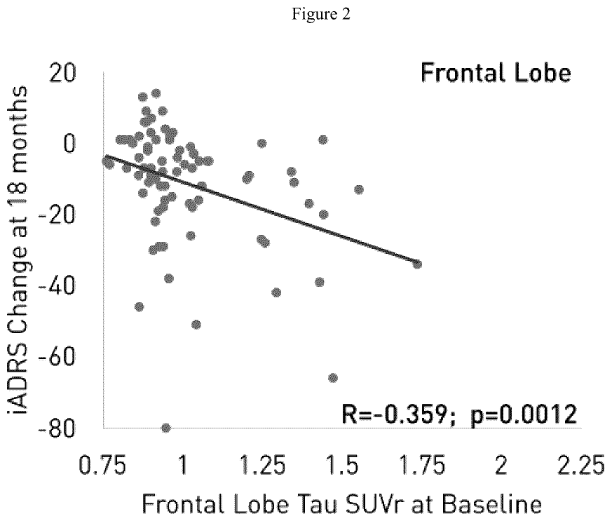

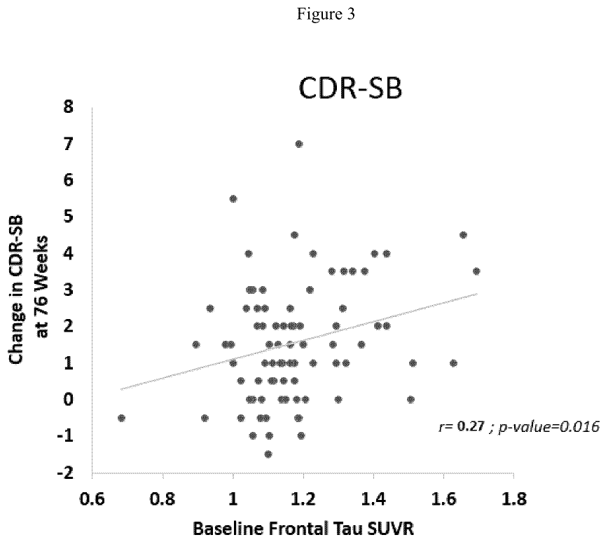

FIGS. 1, 2 and 3 demonstrate a lower cognitive decline associated with lower tau burden at baseline. Additionally, FIGS. 1, 2 and 3 demonstrate heterogeneity in cognitive decline among patients determined to have higher tau burden (e.g., greater then SUVR of about 1.4) at baseline. FIG. 1 shows global tau burden at baseline vs. iADRS change over 18 months. FIG. 2 shows frontal lobe tau burden at baseline vs. iADRS change over 18 months. FIG. 3 shows frontal lobe tau burden at baseline vs. CDR-SB change over 76 weeks.

### Example 3: Treatment of Subject Identified as Having High Neurological Tau Burden

Subjects may be determined, at baseline, to have a high neurological tau burden according to methods as described herein, including PET imagine, including the use of flortaucipir, as well as human pTau217 assessment. Neurological tau burden may be assessed globally, or based on a regional lobal burden, for example posterior-lateral temporal lobe, occipital lobe, parietal lobe and/or frontal lobe. Patients determined to have a high neurological tau burden may be treated with an N3pG antibody described herein and according to dose regimens as described herein.

Additionally, subjects, at baseline, may be cognitively assessed by a manner as described herein including by one of more of ADAS-Cog, iADL, CDR-SB, MMSE, APOE-4 genotyping and/or iADRS. Following treatment with an N3pG Ab described herein, and according to dose regimens as described herein, subjects may be cognitively reassessed, for example at 26 weeks, 52 weeks, 78 weeks, or 104 weeks. Patients demonstrating slow, or not rapid, cognitive decline including patients determined as having a high neurological tau burden may continue to be treated with an N3pG antibody described herein.

Sequences (Underlined portions indicate CDRs)

SEQ ID NO: 1; Light Chain Variable Region (LCVR)
DIVMTQTPLSLSVTPGQPASISCKSSQSLLYSRGKTYLNWLLQKPGQSPQLLIYAVSKLDS
GVPDRFSGSGSGTDFTLKISRVEAEDVGVYYCVQGTHYPFTFGQGTKLEIK
SEQ ID NO: 2; Heavy Chain Variable Region (HCVR)
QVQLVQSGAEVKKPGSSVKVSCKASGYDFTRYYINWVRQAPGQGLEWMGWINPGSGN
TKYNEKFKGRVTITADESTSTAYMELSSLRSEDTAVYYCAREGITVYWGQGTTVTVSS
SEQ IS NO: 3; Light Chain (LC)
DIVMTQTPLSLSVTPGQPASISCKSSQSLLYSRGKTYLNWLLQKPGQSPQLLIYAVSKLDS
GVPDRFSGSGSGTDFTLKISRVEAEDVGVYYCVQGTHYPFTFGQGTKLEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ IS NO: 4; Heavy Chain (HC)
QVQLVQSGAEVKKPGSSVKVSCKASGYDFTRYYINWVRQAPGQGLEWMGWINPGSGN
TKYNEKFKGRVTITADESTSTAYMELSSLRSEDTAVYYCAREGITVYWGQGTTVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPG
SEQ ID NO: 5; Light Chain Complementarity Determining Region 1 (LCDR1)
KSSQSLLYSRGKTYLN
SEQ ID NO: 6; Light Chain Complementarity Determining Region 2 (LCDR2)
AVSKLDS
SEQ ID NO: 7; Light Chain Complementarity Determining Region 3 (LCDR3)
VQGTHYPFT
SEQ ID NO: 8; Heavy Chain Complementarity Determining Region 1 (HCDR1)
GYDFTRYYIN
SEQ ID NO: 9; Heavy Chain Complementarity Determining Region 2 (HCDR2)
WINPGSGNTKYNEKFKG
SEQ ID NO: 10; Heavy Chain Complementarity Determining Region 3 (HCDR3)
EGITVY
SEQ ID NO: 11; Nucleotide Sequence for SEQ ID NO: 1; Light Chain Variable Region (LCVR)
GATATTGTGATGACTCAGACTCCACTCTCCCTGTCCGTCACCCCTGGACAGCCGGCC
TCCATCTCCTGCAAGTCAAGTCAGAGCCTCTTATATAGTCGCGGAAAAACCTATTTG
AATTGGCTCCTGCAGAAGCCAGGCCAATCTCCACAGCTCCTAATTTATGCGGTGTCT
AAACTGGACTCTGGGGTCCCAGACAGATTCAGCGGCAGTGGGTCAGGCACAGATTT
CACACTGAAAATCAGCAGGGTGGAGGCCGAAGATGTTGGGGTTTATTACTGCGTGC
AAGGTACACATTACCCATTCACGTTTGGCCAAGGGACCAAGCTGGAGATCAAA
SEQ ID NO. 12; Nucleotide Sequence for SEQ ID NO: 2; Heavy Chain Variable Region
(HCVR)
CAGGTGCAGCTGGTGCAGTCTGGGGCTGAGGTGAAGAAGCCTGGGTCCTCAGTGAA
GGTTTCCTGCAAGGCATCTGGTTACGACTTCACTAGATACTATATAAACTGGGTGCG
ACAGGCCCCTGGACAAGGGCTTGAGTGGATGGGATGGATTAATCCTGGAAGCGGTA
ATACTAAGTACAATGAGAAATTCAAGGGCAGAGTCACCATTACCGCGGACGAATCC
ACGAGCACAGCCTACATGGAGCTGAGCAGCCTGAGATCTGAGGACACGGCCGTGTA
TTACTGTGCGAGAGAAGGCATCACGGTCTACTGGGGCCAAGGGACCACGGTCACCG
TCTCCTCA
SEQ ID NO. 13; Nucleotide Sequence for SEQ ID NO: 3; Light Chain (LC)
GATATTGTGATGACTCAGACTCCACTCTCCCTGTCCGTCACCCCTGGACAGCCGGCC
TCCATCTCCTGCAAGTCAAGTCAGAGCCTCTTATATAGTCGCGGAAAAACCTATTTG
AATTGGCTCCTGCAGAAGCCAGGCCAATCTCCACAGCTCCTAATTTATGCGGTGTCT
AAACTGGACTCTGGGGTCCCAGACAGATTCAGCGGCAGTGGGTCAGGCACAGATTT
CACACTGAAAATCAGCAGGGTGGAGGCCGAAGATGTTGGGGTTTATTACTGCGTGC
AAGGTACACATTACCCATTCACGTTTGGCCAAGGGACCAAGCTGGAGATCAAACGA
ACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAG
CAGGACAGCAAGGACAGCACCTACAGCCTCAGCAGCACCCTGACGCTGAGCAAAGC
AGACTACGAGAAACACAAAGTCTACGCCTGCGAAGTCACCCATCAGGGCCTGAGCT
CGCCCGTCACAAAGAGCTTCAACAGGGGAGAGTGC
SEQ ID NO. 14; Nucleotide Sequence for SEQ ID NO: 4; Heavy Chain (HC)
CAGGTGCAGCTGGTGCAGTCTGGGGCTGAGGTGAAGAAGCCTGGGTCCTCAGTGAA
GGTTTCCTGCAAGGCATCTGGTTACGACTTCACTAGATACTATATAAACTGGGTGCG
ACAGGCCCCTGGACAAGGGCTTGAGTGGATGGGATGGATTAATCCTGGAAGCGGTA
ATACTAAGTACAATGAGAAATTCAAGGGCAGAGTCACCATTACCGCGGACGAATCC
ACGAGCACAGCCTACATGGAGCTGAGCAGCCTGAGATCTGAGGACACGGCCGTGTA
TTACTGTGCGAGAGAAGGCATCACGGTCTACTGGGGCCAAGGGACCACGGTCACCG
TCTCCTCAGCCTCCACCAAGGGCCCATCGGTCTTCCCGCTAGCACCCTCCTCCAAGA
GCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTC
CAGCAGCTTGGGCACCCAGACCTACATCTGCAACGTGAATCACAAGCCCAGCAACA
CCAAGGTGGACAAGAAAGTTGAGCCCAAATCTTGTGACAAAACTCACACATGCCCA
CCGTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTG
CAAGGTCTCCAACAAAGCCCTCCCAGCCCCCATCGAGAAAACCATCTCCAAAGCCA
AAGGGCAGCCCCGAGAACCACAGGTGTACACCCTGCCCCCATCCCGGGACGAGCTG
ACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCCC
CCGTGCTGGACTCCGACGGCTCCTTCTTCCTCTATAGCAAGCTCACCGTGGACAAGA
GCAGGTGGCAGCAGGGGAACGTCTTCTCATGCTCCGTGATGCATGAGGCTCTGCACA
ACCACTACACGCAGAAGAGCCTCTCCCTGTCTCCGGGT
SEQ ID NO: 15; Amino acid sequence for the Light Chain of Solanezumab
DVVMTQSPLSLPVTLGQPASISCRSSQSLIYSDGNAYLHWFLQKPGQSPRLLIYKVSNRFS
GVPDRFSGSGSGTDFTLKISRVEAEDVGVYYCSQSTHVPWTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 16; Amino acid sequence for the Heavy Chain of Solanezumab
EVQLVESGGGLVQPGGSLRLSCAASGFTFSRYSMSWVRQAPGKGLELVAQINSVGNST
YYPDTVKGRFTISRDNAKNTLYLQMNSLRAEDTAVYYCASGDYWGQGTLVTVSSAST
KGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKS
RWQQGNVFSCSVMHEALHNHYTQKSLSLSPGK

1.-7. (canceled)

8. A method of treating, preventing, or retarding the progression of Alzheimer's Disease (AD) in a human subject who has been determined to have i) slow progressing AD cognitive decline or ii) slow progressing AD cognitive decline and/or one or two alleles of APOE e4, comprising administering a therapeutically effective amount of an anti-Aβ antibody.

9. The method of claim 8, wherein the human subject has been determined to have a high neurological tau burden.

10. The method of claim 9, wherein the human subject has been determined to have posterior-lateral temporal lobe tau burden.

11. The method of claim 10, wherein the human subject has been determined to have posterior-lateral temporal lobe and occipital lobe tau burden.

12. The method of claim 11, wherein the human subject has been determined to have posterior-lateral temporal lobe, occipital lobe, and parietal lobe tau burden.

13. The method of claim 12, wherein the human subject has been determined to have posterior-lateral temporal lobe, occipital lobe, parietal lobe, and frontal lobe tau burden.

14. The method of claim 8, wherein the human subject has been determined to have slow progressing AD cognitive decline by one of more of ADAS-Cog, iADL, CDR-SB, MMSE, APOE-4 genotyping, tau levels, P-tau levels and/or iADRS.

15. The method of claim 14, wherein the human subject has been determined to have slow progressing AD cognitive decline by iADRS.

16. The method of claim 15, wherein iADRS has declined by less than 20.

17. The method of claim 15, wherein iADRS has declined by less than 20 over a 6-month period, a 12-month period, an 18-month period, or a 24-month period.

18.-20. (canceled)

21. The method of claim 14, wherein the human subject has been determined to have slow progressing AD cognitive decline by APOE-4 genotyping.

22. The method of claim 21, wherein the human subject has been determined to be APOE-4 heterozygous or APOE-4 homozygous negative.

23. (canceled)

24. The method of claim 14, wherein the human subject has been determined to have slow progressing AD cognitive decline by MMSE.

25. The method of claim 24, wherein the human subject has been determined to have MMSE of above 27.

26. The method of claim 24, wherein MMSE has declined by less than 3.

27. The method of claim 24, wherein MMSE has declined by less than 3 over a 6-month period, a 12-month period, an 18-month period, or a 24-month period.

28.-30. (canceled)

31. The method of claim 9, wherein the human subject has been determined to have a high neurological tau burden by neurological PET imaging.

32. The method of claim 31, wherein the human subject has been determined to have high neurological tau burden by neurological PET imaging above 1.46 SUVr.

33. The method of claim 9, wherein the human subject has been determined to have high neurological tau burden by quantification of human tau phosphorylated at threonine at residue 217 (“hTau-pT217”).

34. The method of claim 33, wherein hTau-pT217 is quantified in a biological sample of the human subject.

35. The method of claim 34, wherein the biological sample is cerebral spinal fluid.

36. The method of claim 34, wherein the biological sample is one of blood, plasma, or serum.

37. The method of claim 8, wherein the anti-Aβ antibody comprises an anti-N3pG Aβ antibody.

38. The method of claim 37, wherein the anti-N3pG Aβ antibody comprises a light chain variable region (LCVR) and a heavy chain variable region (HCVR), wherein the LCVR and HCVR are selected from:
(a) the LCVR comprising the amino acid sequence of SEQ ID NO: 1 and the HCVR comprising the amino acid sequence of SEQ ID NO: 2; or
(b) the LCVR comprising the amino acid sequence having at least 95% homology to the amino acid sequence of SEQ ID NO: 1 and the HCVR comprising the amino acid sequence having at least 95% homology to the amino acid sequence of SEQ ID NO: 2.

39. The method of claim 8, wherein said step of administering comprises:
i) administering to the human subject one or more first doses of about 100 mg to about 700 mg of an anti-N3pG Aβ antibody, wherein each first dose is administered once about every four weeks; and
ii) about four weeks after administering the one or more first doses, administering to the human subject one or more second doses of greater than 700 mg to about 1400 mg of the anti-N3pG Aβ antibody, wherein each second dose is administered once about every 4 weeks.

40. The method of claim 39, wherein the human subject is administered the first dose once, two times, or three times before administering the second dose.

41. The method of claim 40, wherein the human subject is administered first doses of about 700 mg.

42. The method of claim 39, wherein the human subject is administered one or more second doses of about 800 mg, about 900 mg, about 1000 mg, about 1100 mg, about 1200 mg, about 1300 mg, or about 1400 mg.

43. The method of claim 39, wherein the human subject is administered one or more second doses of about 1400 mg.

44. The method of claim 39, wherein the anti-N3pGlu Aβ antibody is administered to the human subject for a duration of up to 72 weeks.

45. The method of claim 8, wherein the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy.

46. The method of claim 8, wherein the human subject is an early symptomatic AD patient.

47. The method of claim 46, wherein the human subject has prodromal AD and/or mild dementia due to AD.

48.-53. (canceled)

54. A method of treating or preventing a disease characterized by amyloid beta (Aβ) deposits in the brain of a human subject who has been determined to have i) a high neurological tau burden or ii) a high neurological tau burden and/or one or two alleles of APOE e4, comprising administering a therapeutically effective amount of an anti-Aβ antibody.

55. A method of treating or preventing a disease characterized by Aβ deposits in the brain of a human subject who has been determined to have i) a posterior-lateral temporal lobe tau burden or ii) a posterior-lateral temporal lobe tau burden and/or one or two alleles of APOE e4, comprising administering a therapeutically effective amount of an anti-Aβ antibody.


SEQUENCE LISTING (14 sequences)

SEQ ID NO: 1 - protein, 112 aa, Artificial Sequence
  Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 2 - protein, 115 aa, Artificial Sequence
  Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 3 - protein, 219 aa, Artificial Sequence
  Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 444 aa, Artificial Sequence
  Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 16 aa, Artificial Sequence
  Synthetic construct
KSSQSLLYSRGKTYLN

SEQ ID NO: 6 - protein, 7 aa, Artificial sequence
  Synthetic construct
AVSKLDS

SEQ ID NO: 7 - protein, 9 aa, Artificial sequence
  Synthetic construct
VQGTHYPFT

SEQ ID NO: 8 - protein, 10 aa, Artificial sequence
  Synthetic construct
GYDFTRYYIN

SEQ ID NO: 9 - protein, 17 aa, Artificial sequence
  Synthetic construct
WINPGSGNTKYNEKFKG

SEQ ID NO: 10 - protein, 6 aa, Artificial Sequence
  Synthetic construct
EGITVY

SEQ ID NO: 11 - DNA, 336 bp, Artificial Sequence
  Synthetic construct DNA sequence of SEQ ID NO. 1
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaa

SEQ ID NO: 12 - DNA, 345 bp, Artificial Sequence
  Synthetic construct DNA sequence of SEQ ID NO. 2
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctca

SEQ ID NO: 13 - DNA, 657 bp, Artificial Sequence
  Synthetic construct DNA sequence of SEQ ID NO. 3
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 14 - DNA, 1332 bp, Artificial Sequence
  Synthetic construct DNA sequence of SEQ ID NO. 4
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt